(19) United States
(12) Patent Application Publication
(10) Pub. No.: US 2024/0350052 A1
(43) Pub. Date: Oct. 24, 2024

(54) ARTIFICIAL INTELLIGENCE (AI) AND MACHINE LEARNING (ML) BASED GRAPHICAL USER INTERFACE (GUI) SYSTEM FOR EARLY DETECTION OF DEPRESSION SYMPTOMS USING FACIAL EXPRESSION RECOGNITION AND ELECTROENCEPHALOGRAM

(71) Applicants: Tanya Das, Glebe (AU); Brown University, Providence, RI (US); Guru Nanak Dev University, Amritsar (IN)

(72) Inventors: Gajendra Kumar, Providence, RI (US); Eric Morrow, Providence, RI (US); Kuldeep Singh, Amritsar (IN); Tanaya Das, Glebe (AU)

(21) Appl. No.: 18/638,079

(22) Filed: Apr. 17, 2024

Related U.S. Application Data
(60) Provisional application No. 63/460,198, filed on Apr. 18, 2023.

(51) Int. Cl. A61B 5/16; A61B 5/00; A61B 5/372; G06V 10/94; G06V 40/16; G16H 50/20

(52) U.S. Cl. CPC A61B 5/165; A61B 5/0077; A61B 5/7267; G06V 10/945; G06V 40/175; G16H 50/20; A61B 5/372; A61B 2576/02

(57) ABSTRACT

An artificial intelligence (AI) and machine learning (ML) based graphical user interface (GUI) system for early detection of depression symptoms using facial expression recognition and electroencephalogram is provided via receiving image data of a biological subject under examination for depression; receiving, from the biological subject, electroencephalogram data; analyzing the image data, via a first machine learning model, to identify an emotional state conveyed by a face of the biological subject; analyzing the electroencephalogram data, via a second machine learning model, to identify a severity level of depression in the biological subject; and providing the emotional state identified by the first machine learning model and the severity level identified by the second machine learning model to a graphical user interface.

### CROSS-REFERENCES TO RELATED APPLICATIONS

The present disclosure claims the benefit of U.S. Provisional Patent Application No. 62/195,711 entitled “ARTIFICIAL INTELLIGENCE (AI) AND MACHINE LEARNING (ML) BASED GRAPHICAL USER INTERFACE (GUI) SYSTEM FOR EARLY DETECTION OF DEPRESSION SYMPTOMS USING FACIAL EXPRESSION RECOGNITION AND ELECTROENCEPHALOGRAM” and filed on 2023 Apr. 18, which is incorporated herein by reference in its entirety.

### TECHNICAL FIELD

The present disclosure relates to a software tools to improve the efficiency and speed at which computers are able to detect systems of depression via facial expression recognition and electroencephalograms.

### SUMMARY

The present disclosure provides artificial intelligence (AI) and machine learning (ML) based graphical user interface (GUI) system for early detection of depression symptoms using facial expression recognition and electroencephalogram.

In various embodiments, systems, methods, and instructions embodied in computer readable storage media may provide for receiving image data from video of a biological subject under examination for depression; analyzing the image data, via a first machine learning model, to identify an emotional state conveyed by a face of the biological subject; in response to identifying the emotional state as an emotion indicative of depression in the biological subject, requested electroencephalogram data for the biological subject; receiving, from the biological subject, the electroencephalogram data; analyzing the electroencephalogram data, via a second machine learning model, to identify a severity level of depression in the biological subject; and providing the emotional state identified by the first machine learning model and the severity level identified by the second machine learning model to a graphical user interface.

In some such embodiments, the first machine learning model is trained as a random forest model.

In some such embodiments, the second machine learning model is trained as a random forest model.

In some such embodiments, the emotional state is identified as one of the group consisting of: anger; disgust; fear; happiness; sadness; and neutral.

In some such embodiments, the emotional state identified by the first machine learning model and the severity level identified by the second machine learning model are provided to a graphical user interface in real-time.

In some such embodiments, the image data are provided as one of still images or video images.

In some such embodiments, the operations further include generating a treatment plan for the biological subject based on the severity level identified by the second machine learning model.

In some such embodiments, the severity level of depression is identified as one of the group consisting of: not depressed; mildly depressed; moderately depressed; and severely depressed.

Additional features and advantages of the disclosed method and apparatuses are described in, and will be apparent from, the following Detailed Description and the Figures. The features and advantages described herein are not all-inclusive and, in particular, many additional features and advantages will be apparent to one of ordinary skill in the art in view of the figures and description. Moreover, it should be noted that the language used in the specification has been principally selected for readability and instructional purposes, and not to limit the scope of the inventive subject matter.

### BRIEF DESCRIPTION OF THE DRAWINGS

FIG. 1 is a flowchart of an example two-stage methodology implemented according to embodiment of the present disclosure.

FIG. 2 is an example method for identification of a person's emotion via facial expression recognition, according to embodiments of the present disclosure.

FIG. 3 is a flowchart of an example method for detection of depression severity level using EEG analysis, according to embodiments of the present disclosure.

FIG. 4 illustrates the input and output flow of the graphical user interface (GUI), according to embodiments of the present disclosure.

FIGS. 5A-5F illustrate example screen shots of a GUI, according to embodiments of the present disclosure.

FIGS. 6A-6D provide tables that identify advantages of the present disclosure over traditional approaches, as determined experimentally when applying embodiments of the present disclosure.

FIG. 7 illustrates a computing device, according to embodiments of the present disclosure.

### DETAILED DESCRIPTION

The present disclosure provides new and innovative systems and methods for the analysis depression in biological subjects to assist physicians in diagnosing the stages of depression with greater ease, speed, and accuracy to allow for the prophylaxis or treatment of depression in the biological subject via an interactive Graphical User Interface (GUI) tool.

Major depressive disorder (MDD) is a serious detrimental mental condition affecting the daily activities of life. MDD is one of the leading causes of disability among psychological disorders, yet MDD remains widely undiagnosed and untreated due to lack of sensitive and reliable diagnostic tools and methods. MDD is generally characterized by depressed mood (experienced with feeling sad, irritable, empty) or a loss of interest in daily activities or anhedonia accompanied by sleep disruptions, appetite changes, poor focus, lack of energy, and low self-esteem. MDD caused moderate to severe complications, from comorbid anxiety disorders to high risk of suicide. As of 2019, 280 million individuals have been reported to suffer from MDD globally, including 23 million children and adolescents. Incidence has increased by 28% since the Covid-19 pandemic and projected to increase by several fold due to improvement of diagnostic tools.

Conventionally, severity of depression is assessed by two rating scales (i) the Patient Health Questionnaire-9 (PHQ-9); a standardized self-rating scale and (ii) the Hamilton Rating Scale for Depression (HAM-D); a clinician-administered rating scale. Other diagnostic assessments include Montgomery-Asberg Depression Rating Scale (MADRS), Beck Depression Inventory (BDI), and Zung Self-Rating Depression Scale (ZSRDS). However, these assessments are time consuming, require a clinician, and are often accompanied with bias. The majority of clinics and hospitals do not provide mental health services and refer to specialized clinics. Also, diagnosis of early depression symptoms is inconsistent, non-sensitive, non-reliable, and prognosis is ambiguous, which delays the initiation of therapy/medication.

The present disclosure provides to a designed artificial intelligence (AI) tool with a graphical user interface (GUI) to assist clinicians to diagnose the depression stage (mild, moderate, or severe) in a biological subject. The AI tool is implemented for depression detection using emotion recognition and electroencephalography (EEG). In practice, emotion facial recognition using machine learning algorithms has shown an accuracy of 81% to detect whether a person is depressed or not. Multichannel EEG with spectral-spatial feature extraction has successfully classify depression symptoms in practice with an accuracy of 80% using SVM method. The present disclosure combines these two biomarkers of depression (emotion facial recognition and EEG) and achieves, in practice, an accuracy of more than 90% for the detection of depression.

Further, the present disclosure provides a GUI for real-time depression detection, which visualizes the results instantaneously to help the clinicians for prompt diagnosis and initiation of treatment/medication for the appropriate level of depression.

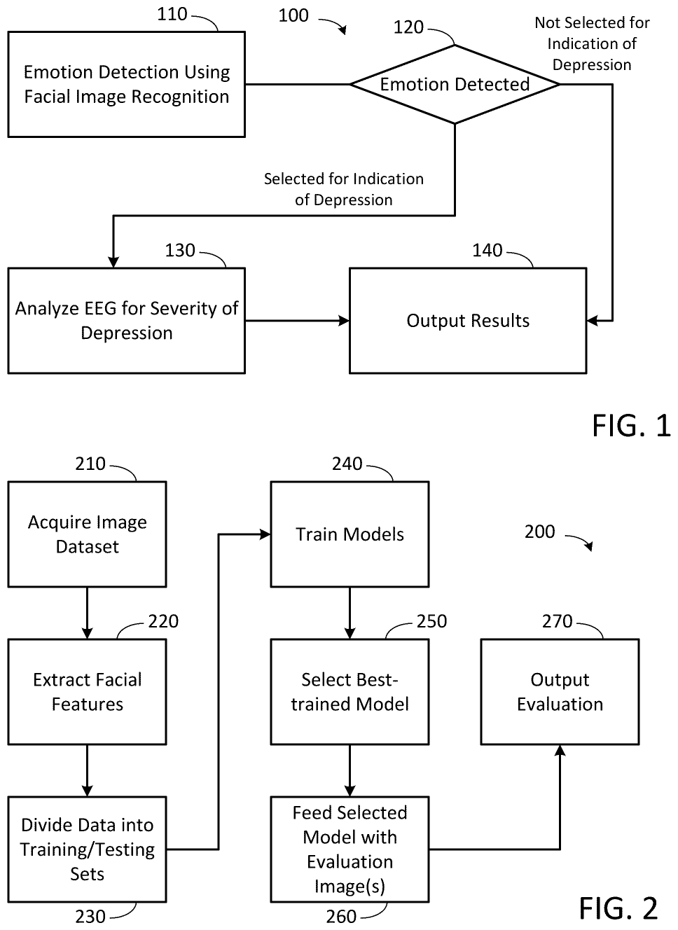

FIG. 1 is a flowchart of an example two-stage methodology 100 implemented according to embodiment of the present disclosure. At block 110, facial image recognition us used to detect depression in a person via emotions displayed by that person in one or more images. The identification of these emotions is accomplished by recognizing the person's facial expression. Such emotions for identification include at least one of: anger, disgust, fear, happiness, sadness, and neutral as depressed facial expressions, while surprise was chosen as a non-depressed facial expression. To correctly recognize the emotions and detect the depression state, the present two-stage methodology involves three operations for each stage: data pre-processing, feature extraction, and model training. Discussion of facial expression is provided with respect to method 200 described in relation to FIG. 2.

At block 120, when an emotion selected for indication of depression is detected, such as anger, disgust, fear, happiness, neutrality, or sadness, the recognition is taken as a physical sign of depression in that person, and method 100 proceeds to block 130. Otherwise, if the emotion recognized is not selected for indication of depression, such as with surprise, the person is considered as not depressed and does not undergo the EEG analysis set forth in block 130, and method 100 may proceed to block 140 with a result that the person is not diagnosed with depression.

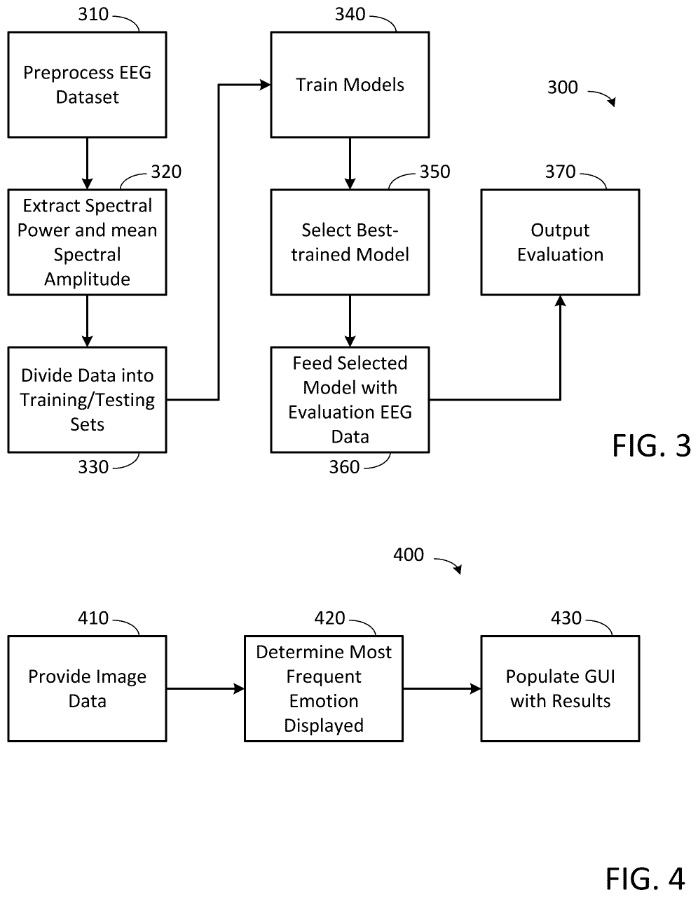

At block 130, an EEG is performed on the person and the EEG of that person is analyzed to determine the depression severity level. Discussion of EEG analysis is provided with respect to method 300 described in relation to FIG. 3.

At block 140, a GUI is populated with results of the analysis. In various embodiments, the GUI is provided with the emotional state identified from the facial analysis and the severity level identified from the EEG data, which may be populated in various frames, windows, or sub-GUIs to provide an easy to understand output of the analyses, and the output may be updated in real-time when real-time image or EEG are provided. In some embodiments, the system may generate (or loo up) a treatment plan for the biological subject based on the severity level identified from the EEG data, which may also be output by the GUI.

FIG. 2 is an example method 200 for identification of a person's emotion via facial expression recognition, according to embodiments of the present disclosure. Method 200 begins at block 210, where an image dataset is acquired and pre-processed for resizing each image into a desired image resolution, and performing any color correction (e.g., standardizing to grayscale). In various embodiments, the images are resized by cropping to a desired size, padding to a desired size, stretching to a desired size, or compressing to a desired size, having a predefined number of pixels in length and width.

Anger, disgust, fear, happiness, sadness, and neutral emotions were chosen as emotions indicative of depressed facial expressions, while surprise was chosen as non-depressed facial expression. People suffering from depression often exhibit angry expressions characterized by furrowed brows and tightened facial muscles, indicative of unresolved anger and irritability. Likewise, expressions of disgust, such as a raised upper lip, narrowed eyes, or a wrinkled nose, may also be observed in those experiencing depression. In some cases, facial expressions of fear, including widened eyes, raised eyebrows, or a tense jaw, may be present even in the absence of an immediate threat.

The inclusion of happiness as a depressed emotion stems from the recognition that individuals with depression can still display moments of genuine happiness even if the expression is transient and inconsistent. These moments are reflected in facial expressions such as joy, smiling, brightened eyes, and relaxed facial muscles, which can quickly transition back to a more neutral or sad expressions.

Sad facial expressions, characterized by a downturned mouth, drooping eyelids, and a lack of vibrancy, emerge as prevalent in individuals struggling with depression. Additionally, neutral expressions, with minimal changes and a lack of facial expression, are also considered as a depression symptom due to emotional numbness or detachment.

Surprise, however, is chosen as a non-depressed emotion because surprise arises in response to unexpected stimuli, involving a momentary shift in attention and physiological arousal. In contrast, individuals experiencing depression display a diminished emotional reactivity, resulting in reduced responsiveness to positive or rewarding stimuli, which impacts the ability of those persons to experience surprise fully. Moreover, when analyzing single images of individuals' facial expressions, facial landmarks associated with the expression of surprise are used for identification. Raised eye-brows, widened eyes showing possible dilation, a change in the width of the nose nostril, and an open mouth represent the characteristic expressions used to identify surprise.

It is worth mentioning that emotions are intricate and context-dependent, and the expression or experience of surprise alone cannot distinctly differentiate between healthy and depressed individuals. While surprise might be more common or prevalent in certain contexts among healthy individuals (due to novelty or unexpected positive events), emotions are multifaceted and not isolated to one single expression or criterion. However, to create a more sophisticated and reliable understanding of emotional differences between healthy individuals and those with depression, a wide array of emotions, combinations of emotions, temporal patterns, contextual factors, and individual variations, are considered rather than focusing on a single emotion as a defining factor. This multifaceted approach helps in developing more nuanced and accurate methods for emotion recognition in the context of mental health assessment.

At block 220, facial features are extracted from the images obtained from video. For example, the MediaPipe face mesh pipeline or other machine learning based systems may be used to extract facial features to describe different emotions. The facial landmark features provide information about the specific shape and position of facial features, such as the eyes, eyebrows, nose, mouth, and chin. These landmark features allow for a more detailed analysis of subtle facial movements and expressions, which allows for accurately detecting and recognizing emotions. Instead of working with the entire pixel data of an image, extracting and analyzing the positions of facial landmarks simplifies the input and focuses on the most relevant features for emotion recognition. Furthermore, facial landmarks are more robust and stable compared to other features, as facial landmarks can be accurately detected even in the presence of variations such as head movements leading to improved accuracy and effectiveness in emotion recognition systems.

In various embodiments, these landmarks are 3D landmarks that consist of width (x), height (y), and depth (z) coordinates. For example, 3D landmarks may be extracted for the lips, left eye, right eye, left eyebrow, right eyebrow, outline of the entire face, and 3D mesh landmarks for the irises.

Extraction of the landmarks by the pipeline may involve work of two deep neural network models: a face detection model that computes regions of the face while operating on the entire image, and a 3D face landmark regression model that operates on those detected face regions and predicts the 3D surface. The face detection model acquires an accurate facial region of interest from the full image as input to the landmark model. To extract the landmarks, the face landmark model uses transfer learning to predict the 3D landmark coordinates and 2D semantic contours on synthetic rendered data and annotated real-world data, respectively.

At block 230, the data are divided into training and testing sets (e.g., and 80/20 split). To address class imbalance, a Synthetic Minority Oversampling Technique with Edited Nearest Neighbors (SMOTE/ENN) analysis to resample the image data, equalizing the number of samples for each class. The training and testing datasets are then standardized to have a mean of zero and a standard deviation of one.

At block 240, the resultant data from block 230 are fed into machine learning classifiers (or deep leaning algorithms) such as random forest, decision tree, extremely randomized trees (extra trees), histogram gradient boosting (HGB), extreme gradient boosting (XGBoost), and a convolutional neural network (CNN). The various models are trained according to the selected training and testing data sets, and various set-up parameters chosen by the user.

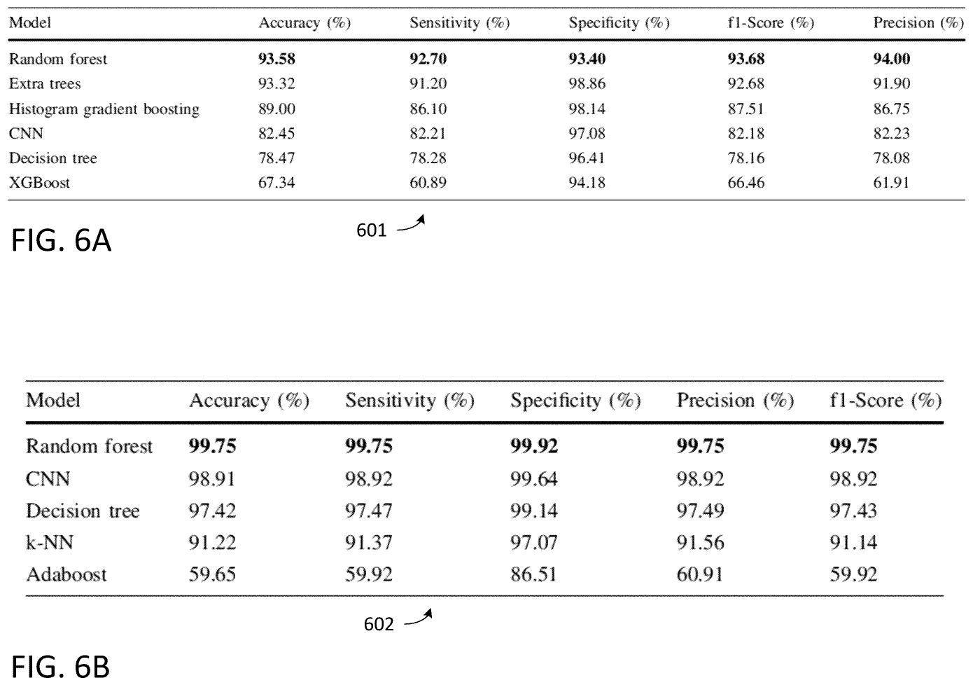

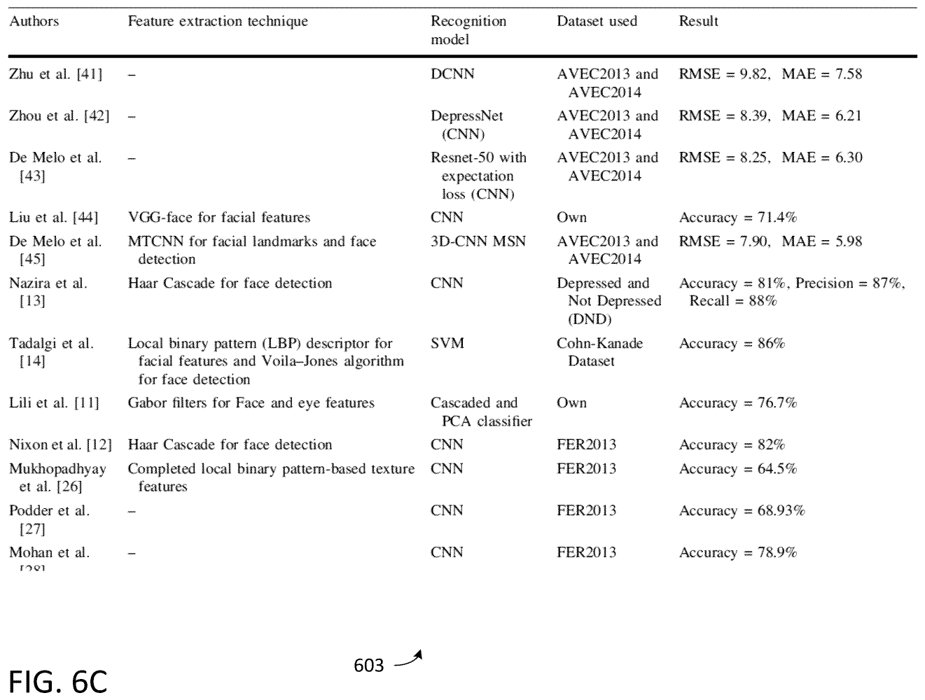

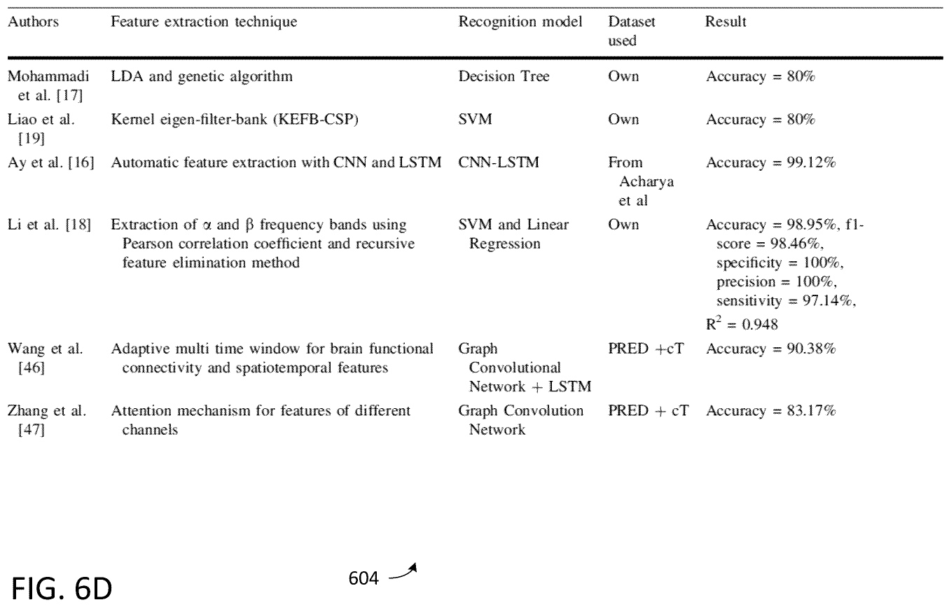

At block 250, the best-performing classifier trained per block 240 that identifies the emotion classes most correctly is selected. To identify emotions and detect the presence of depression symptoms through facial expression recognition, the trained models (e.g., random forest, decision tree, extra trees, HGB, XGBoost, CNN model) are evaluated against one another based on the ability to correctly identify emotion classes, namely anger, disgust, fear, happiness, neutral, sadness, and surprise, using the aforementioned metrics. Table 601 in FIG. 6A presents the metrics values obtained during testing of the models.

To evaluate the performance of the classification models, metrics such as accuracy, sensitivity, specificity, precision, and f1-score may be used, which are defined by Formulas 1-5, respectively, where TP, TN, FP, and FN denote true positive, true negative, false positive, and false negative predictions, respectively, obtained from confusion matrices of the given machine learning classifiers.

$$Accuracy = \frac{( T P + T N )}{( T P + T N + F P + F N )} \qquad Formula 1$$

$$Precision = \frac{T P}{( T P + F P )} \qquad Formula 2$$

$$Sensitivity = \frac{T P}{( T P + F N )} \qquad Formula 3$$

$$Specificity = \frac{T N}{( T N + F P )} \qquad Formula 4$$

$$f 1 score = 2 * \frac{( Precision * Sensitivity )}{( Precision + Sensitivity )} \qquad Formula 5$$

One of skill in the art will appreciate that particular results for a given model may be the result of the inputs used to train the models and the variables set in constructing the models, however, experimentally, the various model types displayed different benefits during experimentation, and a user may select different models based on different goals. For example, experimental results indicate that random forest achieved the highest accuracy of 93.58%, closely followed by extra trees with an accuracy of 93.32%. Decision tree, HGB, and CNN models demonstrated accuracy ranging from 78 to 89%, while XGBoost displayed the lowest accuracy at 67.34%. Among all the models, random forest exhibited the highest sensitivity at 92.70%, f1-score at 93.68%, and precision at 94.00%. Extra trees showed a sensitivity of 91.20%, f1-score of 92.68%, and a precision of 91.90% next to random forest. However, extra trees achieved the highest specificity at 98.86%, while random forest exhibited a specificity of 93.40%.

Generally during experimentation random forest has been found to out-perform other models in terms of accuracy, sensitivity, f1-score, and precision, closely followed by extra trees. Among all the models tested, random forest exhibited maximum sensitivity values of 90.9%, 93.71%, 95.19%, 92.46%, and 92.46% for the anger, fear, happiness, neutral, and sadness classes, respectively. The random forest model also achieved optimally high values of 92.11% and 92.12% for the disgust and surprise classes, respectively. Similarly, the random forest model showed maximum specificity values of 98.9%, 99.99% and 98.98% for the anger, disgust, and surprise classes, respectively, along with optimal values of more than 98% for other classes. Moreover, the random forest model demonstrated impactful performance in terms of maximum precision values for all classes compared to other models. In the same regard, the random forest model also exhibited impressive performance in terms of the f1-score, achieving highest values for all emotion classes, except for disgust class, for which the random forest model achieved a satisfactorily high f1-score of 95.24%.

At block 260, the selected classifier is fed still images and videos of a biological subject who is not known to be affected by depression. The identification of emotion can use the analysis of both images and videos in order to accurately determine whether an individual is experiencing depression or not. Although emotion identification models may be trained using an image dataset to predict emotions from image samples per block 240, the same models can also be employed to detect emotions from a person's video. This is attributable to the fact that a video essentially comprises a sequence of images or frames, and the emotional state of each frame or image can be ascertained by implementing the trained model on each frame or image within the video.

At block 270, the classifier outputs a determination of whether the evaluated images or videos of the biological subject.

FIG. 3 is a flowchart of an example method 300 for detection of depression severity level using EEG analysis, according to embodiments of the present disclosure.

Method 300 begins a block 310, where the EEG dataset is pre-processed for removal of noise and extraction of features from the depression severity classes. Pre-processing includes filtering, segmentation, and feature extraction. Filtering may be performed using a second-order Butterworth band pass filter with a frequency range of 0.1-250 Hz and a 50-Hz notch filter to remove artefacts and power line interference, respectively.

Following filtering, the resultant EEG signals are segmented into 5 second segments using non-overlapping temporal windows to handle the non-stationary nature of the signals.

EEG segmentation is followed by spectral domain transformation, where time-domain EEG signals are transformed into the frequency domain, for example, by using Fast Fourier Transform (FFT) technique. The frequency domain re-classification allows for accurate prediction of the severity of classes.

The EEG is separated into multiple spectral bands, viz. delta (0.1-4 Hz), theta (4-8 Hz), alpha (8-12 Hz), beta (12-30 Hz), low gamma-0 (30-50 Hz), low gamma-1 (30-50 Hz), high gamma-0 (70-100 Hz), and high gamma-1 (100-250 Hz) brain waves. In some embodiments, the gamma band is further separated along with other spectral bands by dividing the bands into several sub-bands. This approach is based on the understanding that gamma-band oscillations contribute to the precise detection of depression status in depression patients. Moreover, the diverse spectral features extracted from the high-frequency gamma sub-bands possess enhanced descriptive capabilities, which aid in distinguishing the severity levels during depression detection.

At block 320, spectral power and mean spectral amplitude are extracted for each spectral band. Mathematically, mean spectral amplitude(S) and spectral power (P) are expressed in μV/Hz and μV²/Hz by Formula 1 and Formula 2 (below), respectively, for an N-point signal x(n) having discrete Fourier Transform X(k) for a specific spectral band, where X*(k) represents the complex conjugate of X(k) in the frequency domain.

$$S ( k ) = \frac{1}{N} {\sum }_{k = 0}^{N - 1} X ( k ) \qquad Formula 1$$

$$P = \frac{1}{N} {\sum }_{k = 0}^{N - 1} X ( k ) {X}^{*} ( k ) \qquad Formula 2$$

At block 330, the resultant data are dividing into a training set and a testing data set (e.g., via an 80/20 split) to train various classifiers.

At block 340, the data are used to train the various classifiers/models according to the selected training and testing data sets, and various set-up parameters chosen by the user. The classifiers trained may include random forest, decision tree, k-nearest neighbors (k-NN), adaptive boosting (Ada-Boost), and CNN. AdaBoost is a type of ensemble learning model based on decision trees, while k-NN algorithm produces output based on distance.

At block 350, the best-performing model that identifies the severity of symptoms most correctly is selected. The evaluation involved the use of the metrics described earlier. Table 602 in FIG. 6B presents the metrics values obtained during testing of the models. It is observed that random forest exhibited the highest accuracy (99.75%), sensitivity (99.75%), specificity (99.92%), precision (99.75%), and f1-score (99.75%) among all the models and across all severity levels. CNN followed closely with an accuracy of 98.91%, sensitivity of 98.92%, specificity of 99.64%, precision of 98.92%, and a f1-score of 98.92%. On the other hand, Adaboost achieved the lowest accuracy of 59.65% among all models. These results emphasized the precision and sensitivity of random forest in classifying the severity levels of depression in patients with depression. The random forest model displayed highest sensitivity values of 99.67%, 100%, 99.33%, and 100% for control, mild, moderate, and severe levels, respectively. Similarly, random forest exhibited maximum specificity values of 99.77%, 99.96%, 99.33%, and 100% for the control, mild, moderate, and severe classes compared to other classifiers.

Additionally, the random forest model demonstrated effective performance with highest precision values of 99.35, 99.87, 99.8, and 100% for the control, mild, moderate, and severe classes, respectively. The random forest model also achieved highest f1-score values of exceeding 99% for all depression severity classes among all other models.

At block 360, the selected model is fed evaluation EEG data for a biological subject under analysis.

At block, 370, the model outputs a determination of severity of depressive systems for the biological subject under analysis.

A graphical user interface (GUI) application to work as an AI assistant for one or both of the analyses made per method 200 and method 300 may be provided for a user-friendly approach for output and review of the analyses of the implemented approach to detect depression severity through facial expression recognition and EEG analysis. Integration of the developed artificial intelligence (AI) models with the GUI may be accomplished by deploying the GUI into a web application (e.g., using Streamlit framework or another framework for deploying machine learning and deep learning models). The GUI displays the outcome of the biological subject either having depression or not based on the emotion recognized, and when the biological subject is detected as having depression, displays a detected severity level. The user has the ability to predict the result from both real-time images or video streams and recorded images or video streams.

FIG. 4 illustrates the input and output flow 400 of the GUI, according to embodiments of the present disclosure.

At block 410, the user engages in an emotion identification process by providing an image or video captured by a camera. In the case of image input, the GUI directly processes the image and delivers the corresponding result. However, when dealing with video input, the GUI algorithm divides the video into frames based on the duration and frame count of the video.

At block 420, the GUI determines the most frequently detected emotion among all emotions identified from each frame, considering the most-frequently identified emotion as the primary emotion identified. If the outcome reveals one of the depressed emotion classes, the subject will be prompted to undergo EEG analysis to assess the severity level of depression in the subject.

At block 430, the GUI is populated with the results of the analyses for whether the subject displays a primary emotion associated with depressive symptoms (from the image analysis) and a severity of the depressive systems (from the EEG data), if relevant.

In various embodiments, the GUI provides a multi-section interactive interface starting with a ‘Homepage’ section that displays a welcome message and description of the functionalities provided therein. A ‘Emotion Detection (Image)’ section identifies the emotion from both real-time and stored images and displays the identified emotion, and a ‘Emotion Detection (Video)’ section that too identifies the emotion from both real-time and recorded videos and displays the identified emotion. Another section provides the results of the EEG analysis the detection of depression severity level from EEG data that detected and displayed the result of the EEG analysis. FIG. 9 showcases some screenshots of the GUI application in (a) emotion recognition from real-time video stream and (b) depression severity detection using EEG input.

Detecting the degree of severity in patients with depression mostly involves either facial expression identification or EEG analysis. However, by combing emotion recognition and EEG analysis to detect the severity of depression, various benefits may be realized. To provide a user-friendly way of obtaining the detection results, we developed a GUI that integrates it with an AI model. The developed AI assistant initially provided an indication of the presence of depression symptoms and then proceeded to present the severity level detected. For carrying out the overall process of detection, the underlying mechanism of the GUI involved machine learning models that were trained for the purpose. The models showed high sensitivity, precision, and accuracy in the results.

The emotion identification step provides for a preliminary knowledge of whether the subject exhibits a depression condition or not. Accordingly, detection of depression severity level via EEG analysis may follow facial recognition or may be omitted depending on the primary emotion identified from the image data. The GUI is computationally efficient and allows for the desired detection task's result to be instantaneously displayed in real-time with the analysis or provide a historical record of the analysis. By storing the analysis, a patient may perform an initial emotional review via images and share the results with a clinician, who may be located remotely from the patient, before requesting or being instructed to supply EEG data (which may require travel to a clinical setting).

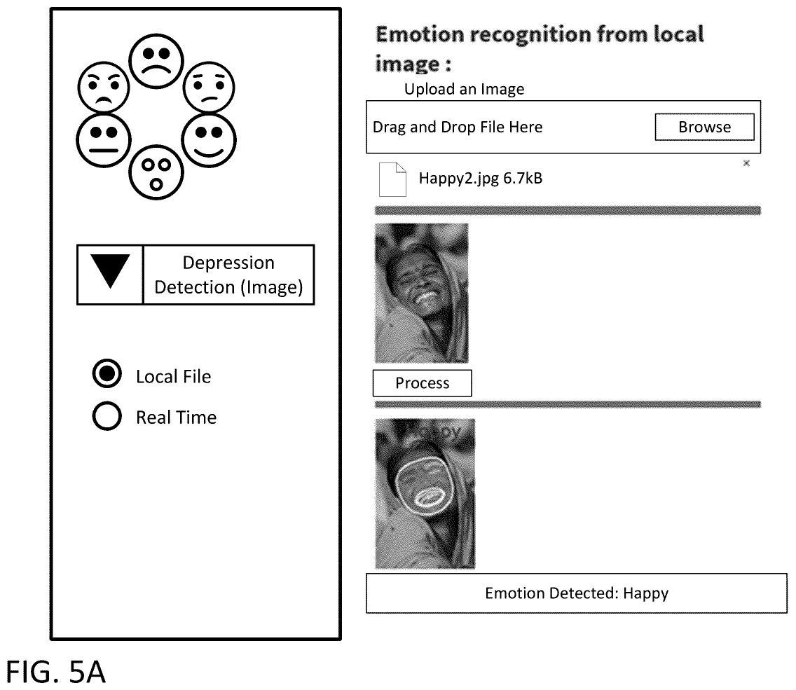

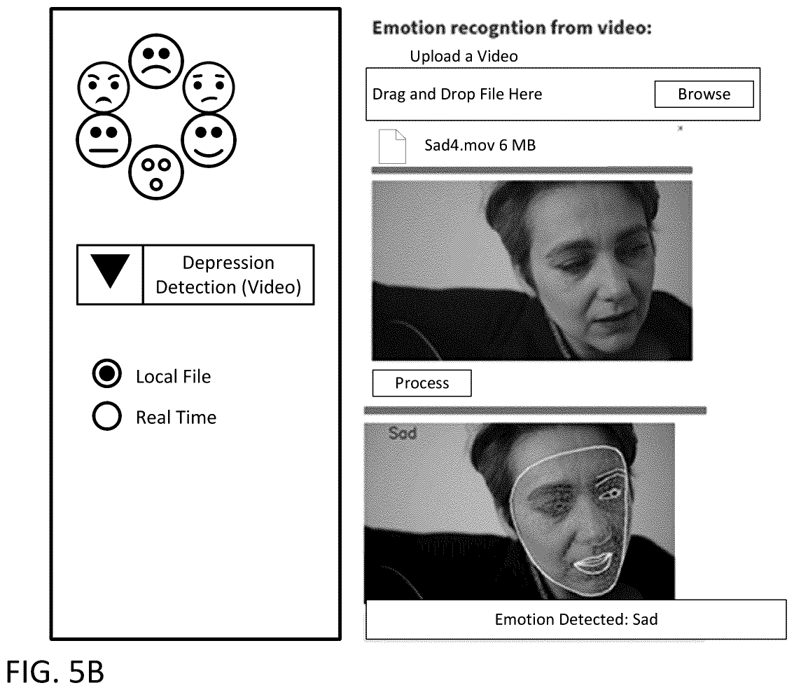

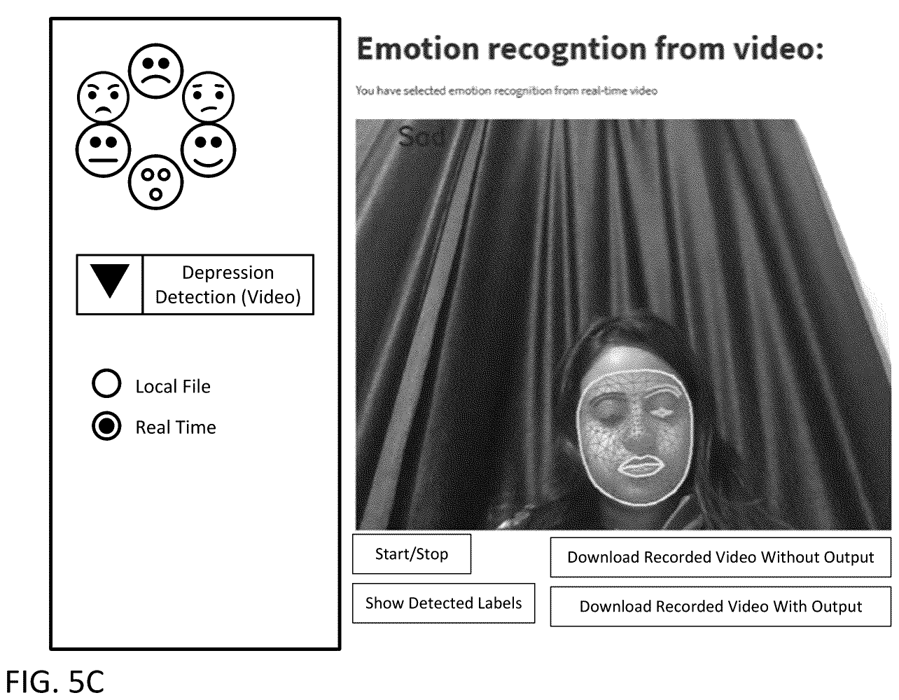

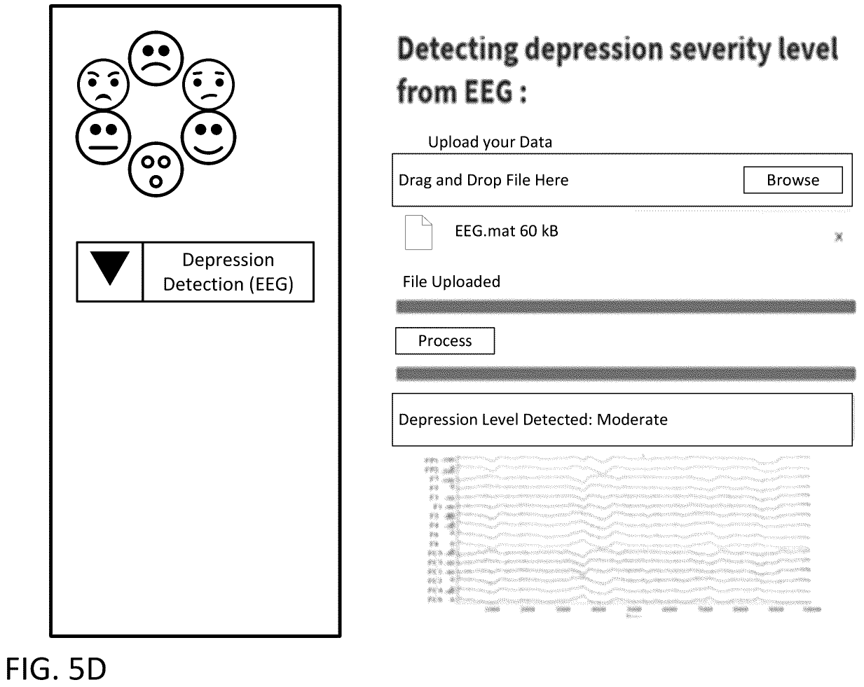

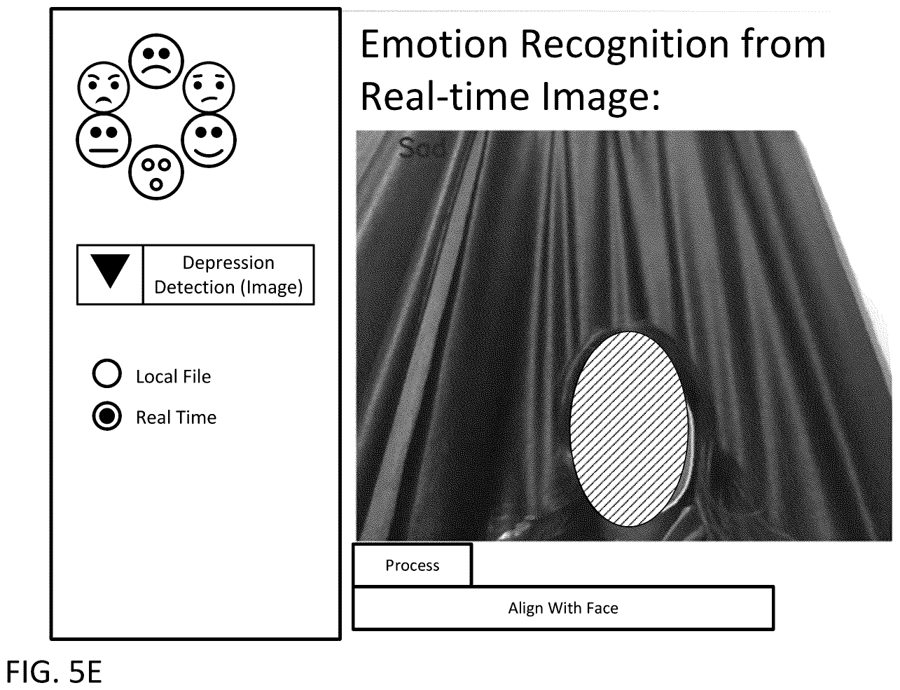

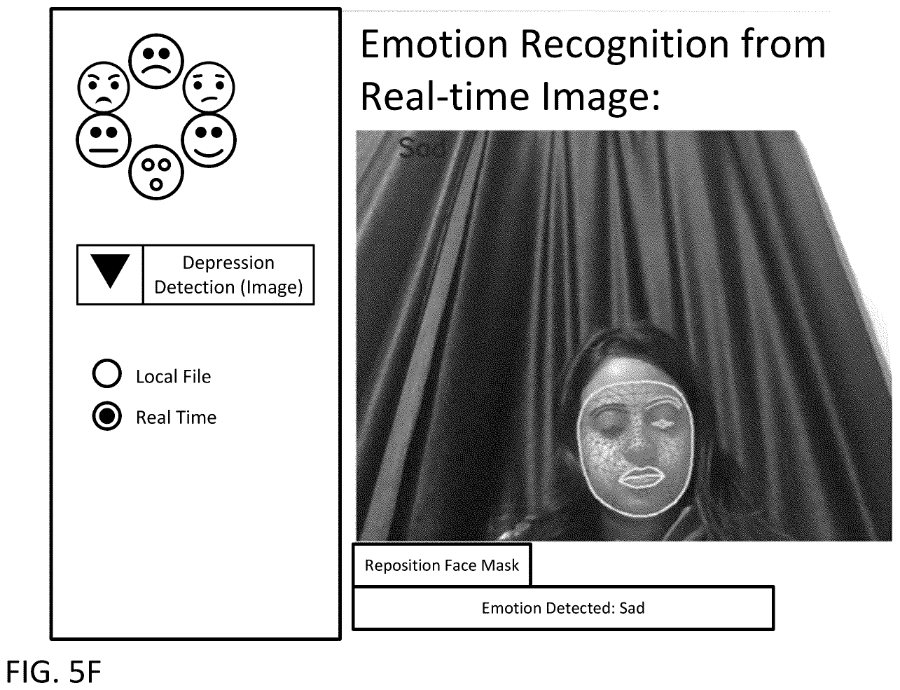

FIGS. 5A-5E illustrate example screen shots 500a-e of a GUI, according to embodiments of the present disclosure. As will be appreciated, various preferences may change the overall appearance of the GUI, as will the data provided by the patient. Accordingly, the screen shots 500a-e are provided as non-limiting examples of how a GUI may be used to provide easily digestible and interactive review of the results of the described emotion recognition system.

Although the described emotion recognition system can accurately identify basic emotions, the reliance on the primary or predominant emotion as an indicator of depression requires increased robustness for medical diagnosis. Uncertainties arise from factors such as the complexity of depression, encompassing a wide range of emotional states, behaviors, and cognitive patterns, with manifestations varying widely among individuals. Additionally, emotional expression is subjective and varies significantly among individuals, contributing to the challenge. The presence of comorbidity and co-occurring symptoms, where depression often coincides with other mental health conditions or physical ailments, further underscores the challenges of identifying depression recognition, potentially overlooking crucial co-occurring symptoms or conditions. In a medical context, the use of basic emotion recognition as a standalone diagnostic tool for depression remains uncertain and insufficient. Instead, facial emotional recognition of depression symptoms can be considered as a supportive, screening, or supplementary tool for mental health professionals when used in conjunction with comprehensive assessments that include interviews, standardized scales, behavioral observations, and other diagnostic criteria.

### Surprising and Unexpected Results

A comparison with the results reported in the use of the described approach for emotion identification using facial expression recognition is presented in Table 603 shown in FIG. 6C. By using different feature extraction techniques and classification algorithms that what have been attempted in existing approaches provides for improvements in overall detection and classification irrespective of different datasets used for the task of emotions classification. The presently described two-tiered approach helps in illustrating the effectiveness of the proposed approach of emotion identification for given feature extraction and classification techniques.

As per table 603, most of the studies presented made use of deep learning algorithms, reporting an accuracy of less than 90%. Studies by Zhu et al., Zhou et al., and De Melo et al. used deep CNN regression techniques to detect depression levels based on the AVEC2013 and AVEC2014 datasets. These studies employed CNN architectures for automated feature extraction, using RMSE and MAE as performance metrics. Further, De Melo et al. used a deep Multiscale Spatiotemporal Network (3D-CNN MSN hybrid architecture) to identify depression severity level using RMSE and MAE as performance metrics. While Liu et al. achieved 71.4% accuracy using VGG-Face algorithm-based feature extraction, previous studies showed less than 83% accuracy in detecting faces using Haar Cascade. Furthermore, studies based of the FER2013 dataset demonstrated that the CNN based approaches provided by Nixon et al. Mukhopadhyay et al., Podder et al., and Mohan et al. showed inferior performances with accuracies of less than 90% for facial expression-based emotion recognition. However, surprising, by extracting 3D facial landmarks as described in the present disclosure, the models were able to achieve more than 90% accuracy in detecting depression through emotion identification with the help of random forest modelling. In addition to the higher accuracy provided by random forest modelling over the traditional CNN algorithms, the use of random forest-based ensemble method provided several additional advantages, including a simpler structure, faster training and classification speeds, and reduced requirements of computational resources as compared to CNN architectures.

Furthermore, table 604 shown in FIG. 6D provides a comparison between related works and the described approach in detecting depression and assessing the severity level of depression through EEG analysis. Table 604 also demonstrates different combinations of feature extraction and classification models in existing approaches for EEG signal analysis-based depression severity irrespective of the utility of different EEG datasets. For example, to detect depression, studies provided by Mohammadi et al. and Liao et al. employed LDA and KEFB-CSP, respectively, for feature extraction using decision tree and SVM models, while Ay et al. used a CNN-LSTM hybrid model, leveraging automatic feature extraction through the CNN and LSTM networks. These studies however obtained an accuracy of less than 99.20%. Another study by Li et al. focused on detecting both depression and the severity levels thereof, with a and b frequency using linear regression. Furthermore, the study provided by Wang et al. used the concept of adaptive multi-time windowing to obtain brain functional connectivity and spatiotemporal features for classification of depressed and non-depressed subjects using graph convolutional network and obtain an accuracy of 90.38%. Similarly, Zhang et al. detected the occurrence of depression using attention mechanism-based graph convolution networks and LSTM models and obtained accuracy of only 83.17%.

In contrast, the present disclosure provides for extracting two features from the EEG data; namely, spectral power and mean spectral amplitude, from eight EEG spectral bands to detect depression severity level using a random forest model. Conventionally, previous studies focused on depression detection from EEG analysis using machine learning and deep learning techniques, with only a few addressing the determination of depression severity levels using EEG. The provided approach achieves a very high accuracy of 99.75% in identifying the depression severity levels, outperforming the traditional methods, which achieved a maximum accuracy of 98.95% in detecting depression. The presently described AI framework exhibits higher accuracy, specificity, sensitivity, precision, and f1-score, and additionally providing the additional advantage of a GUI compared to previous solutions in detecting depression severity levels using both facial expression recognition and EEG analysis.

The present disclosure provides an AI framework with a graphical user interface (GUI) enabled application for the detection and reporting of depression severity. The task is accomplished through facial expression recognition and EEG analysis using machine learning models. Accuracy, specificity, sensitivity, precision, and f1-score were obtained for the implemented models. In various embodiments, random forest was selected as the best among all the models, although other selections may be made in other use cases, although experimentally the values of performance metrics for random forest were highest across all classes in both facial expression recognition and EEG analysis. Random forest showed an accuracy of 93.58% and 99.75% for emotion identification and depression severity detection, respectively, suggesting the reliability of the developed frame-work. The use of EEG with facial expression analysis provided a more accurate identification of patients with depression as well as depression severity levels in those patients.

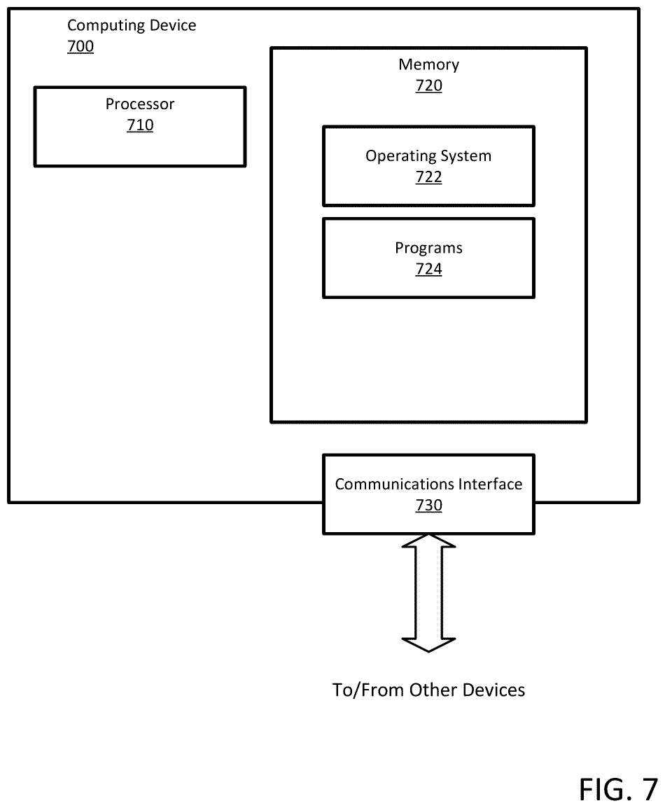

FIG. 7 illustrates a computing device 700, as may be used to provide an artificial intelligence (AI) and machine learning (ML) based graphical user interface (GUI) system for early detection of depression symptoms using facial expression recognition and electroencephalogram, according to embodiments of the present disclosure. The computing device 700 may include at least one processor 710, a memory 720, and a communication interface 730.

The processor 710 may be any processing unit capable of performing the operations and procedures described in the present disclosure. In various embodiments, the processor 710 can represent a single processor, multiple processors, a processor with multiple cores, and combinations thereof.

The memory 720 is an apparatus that may be either volatile or non-volatile memory and may include RAM, flash, cache, disk drives, and other computer readable memory storage devices. Although shown as a single entity, the memory 720 may be divided into different memory storage elements such as RAM and one or more hard disk drives. As used herein, the memory 720 is an example of a device that includes computer-readable storage media, and is not to be interpreted as transmission media or signals per se.

As shown, the memory 720 includes various instructions that are executable by the processor 710 to provide an operating system 722 to manage various features of the computing device 700 and one or more programs 724 to provide various functionalities to users of the computing device 700, which include one or more of the features and functionalities described in the present disclosure. One of ordinary skill in the relevant art will recognize that different approaches can be taken in selecting or designing a program 724 to perform the operations described herein, including choice of programming language, the operating system 722 used by the computing device 700, and the architecture of the processor 710 and memory 720. Accordingly, the person of ordinary skill in the relevant art will be able to select or design an appropriate program 724 based on the details provided in the present disclosure.

The communication interface 730 facilitates communications between the computing device 700 and other devices, which may also be computing devices as described in relation to FIG. 7. In various embodiments, the communication interface 730 includes antennas for wireless communications and various wired communication ports. The computing device 700 may also include or be in communication, via the communication interface 730, one or more input devices (e.g., a keyboard, mouse, pen, touch input device, etc.) and one or more output devices (e.g., a display, speakers, a printer, etc.).

Although not explicitly shown in FIG. 7, it should be recognized that the computing device 700 may be connected to one or more public and/or private networks via appropriate network connections via the communication interface 730. It will also be recognized that software instructions may also be loaded into a non-transitory computer readable medium, such as the memory 720, from an appropriate storage medium or via wired or wireless means.

Accordingly, the computing device 700 is an example of a system that includes a processor 710 and a memory 720 that includes instructions that (when executed by the processor 710) perform various embodiments of the present disclosure. Similarly, the memory 720 is an apparatus that includes instructions that, when executed by a processor 710, perform various embodiments of the present disclosure.

The present disclosure may be understood as a method, comprising: receiving image data of a biological subject under examination for depression; receiving, from the biological subject, electroencephalogram data; analyzing the image data, via a first machine learning model, to identify an emotional state conveyed by a face of the biological subject; analyzing the electroencephalogram data, via a second machine learning model, to identify a severity level of depression in the biological subject; and providing the emotional state identified by the first machine learning model and the severity level identified by the second machine learning model to a graphical user interface.

In some embodiments of the method, the first machine learning model is trained as a random forest model.

In some embodiments of the method, the second machine learning model is trained as a random forest model.

In some embodiments of the method, the emotional state is identified as one of the group consisting of: anger; disgust; fear; happiness; sadness; surprise; and neutral.

In some embodiments of the method, the emotional state identified by the first machine learning model and the severity level identified by the second machine learning model are provided to a graphical user interface in real-time.

In some embodiments of the method, the image data are provided as one of still images or video images.

In some embodiments of the method, the method includes generating a treatment plan for the biological subject based on the severity level identified by the second machine learning model.

In some embodiments of the method, the severity level of depression is identified as one of the group consisting of: not depressed; mildly depressed; moderately depressed; and severely depressed.

Certain terms are used throughout the description and claims to refer to particular features or components. As one skilled in the art will appreciate, different persons may refer to the same feature or component by different names. This document does not intend to distinguish between components or features that differ in name but not function.

As used herein, the term “optimize” and variations thereof, is used in a sense understood by data scientists to refer to actions taken for continual improvement of a system relative to a goal. An optimized value will be understood to represent “near-best” value for a given reward framework, which may oscillate around a local maximum or a global maximum for a “best” value or set of values, which may change as the goal changes or as input conditions change. Accordingly, an optimal solution for a first goal at a given time may be suboptimal for a second goal at that time or suboptimal for the first goal at a later time.

As used herein, various chemical compounds are referred to by associated element abbreviations set by the International Union of Pure and Applied Chemistry (IUPAC), which one of ordinary skill in the relevant art will be familiar with. Similarly, various units of measure may be used herein, which are referred to by associated short forms as set by the International System of Units (SI), which one of ordinary skill in the relevant art will be familiar with.

As used herein, “about,” “approximately” and “substantially” are understood to refer to numbers in a range of the referenced number, for example the range of −10% to +10% of the referenced number, preferably −5% to +5% of the referenced number, more preferably −1% to +1% of the referenced number, most preferably −0.1% to +0.1% of the referenced number.

Furthermore, all numerical ranges herein should be understood to include all integers, whole numbers, or fractions, within the range. Moreover, these numerical ranges should be construed as providing support for a claim directed to any number or subset of numbers in that range. For example, a disclosure of from 1 to 10 should be construed as supporting a range of from 1 to 8, from 3 to 7, from 1 to 9, from 3.6 to 4.6, from 3.5 to 9.9, and so forth.

As used in the present disclosure, a phrase referring to “at least one of” a list of items refers to any set of those items, including sets with a single member, and every potential combination thereof. For example, when referencing “at least one of A, B, or C” or “at least one of A, B, and C”, the phrase is intended to cover the sets of: A, B, C, A-B, B-C, and A-B-C, where the sets may include one or multiple instances of a given member (e.g., A-A, A-A-A, A-A-B, A-A-B-B-C-C-C, etc.) and any ordering thereof. For avoidance of doubt, the phrase “at least one of A, B, and C” shall not be interpreted to mean “at least one of A, at least one of B, and at least one of C”.

As used in the present disclosure, the term “determining” encompasses a variety of actions that may include calculating, computing, processing, deriving, investigating, looking up (e.g., via a table, database, or other data structure), ascertaining, receiving (e.g., receiving information), accessing (e.g., accessing data in a memory), retrieving, resolving, selecting, choosing, establishing, and the like.

Without further elaboration, it is believed that one skilled in the art can use the preceding description to use the claimed inventions to their fullest extent. The examples and aspects disclosed herein are to be construed as merely illustrative and not a limitation of the scope of the present disclosure in any way. It will be apparent to those having skill in the art that changes may be made to the details of the above-described examples without departing from the underlying principles discussed. In other words, various modifications and improvements of the examples specifically disclosed in the description above are within the scope of the appended claims. For instance, any suitable combination of features of the various examples described is contemplated.

Within the claims, reference to an element in the singular is not intended to mean “one and only one” unless specifically stated as such, but rather as “one or more” or “at least one”. Unless specifically stated otherwise, the term “some” refers to one or more. No claim element is to be construed under the provision of 35 U.S.C. § 112 (f) unless the element is expressly recited using the phrase “means for” or “step for”. All structural and functional equivalents to the elements of the various embodiments described in the present disclosure that are known or come later to be known to those of ordinary skill in the relevant art are expressly incorporated herein by reference and are intended to be encompassed by the claims. Moreover, nothing disclosed in the present disclosure is intended to be dedicated to the public regardless of whether such disclosure is explicitly recited in the claims.

What is claimed is:

1. A method, comprising:
receiving image data of a biological subject under examination for depression;
analyzing the image data, via a first machine learning model, to identify an emotional state conveyed by a face of the biological subject;
in response to identifying the emotional state as a emotion indicative of depression in the biological subject, requested electroencephalogram data for the biological subject;
receiving, from the biological subject, the electroencephalogram data;
analyzing the electroencephalogram data, via a second machine learning model, to identify a severity level of depression in the biological subject; and
providing the emotional state identified by the first machine learning model and the severity level identified by the second machine learning model to a graphical user interface.

2. The method of claim 1, wherein the first machine learning model is trained as a random forest model.

3. The method of claim 1, wherein the second machine learning model is trained as a random forest model

4. The method of claim 1, wherein the emotional state is identified as one of the group consisting of:
anger;
disgust;
fear;
happiness;
sadness; and
neutral.

5. The method of claim 1, wherein the emotional state identified by the first machine learning model and the severity level identified by the second machine learning model are provided to a graphical user interface in real-time.

6. The method of claim 1, wherein the image data are provided as one of still images or video images.

7. The method of claim 1, further comprising:
generating a treatment plan for the biological subject based on the severity level identified by the second machine learning model.

8. The method of claim 1, wherein the severity level of depression is identified as one of the group consisting of:
not depressed;
mildly depressed;
moderately depressed; and
severely depressed.

9. A system, comprising:
a processor; and
a memory, including instructions that, when executed by the processor perform operations including:
receiving image data of a biological subject under examination for depression;
analyzing the image data, via a first machine learning model, to identify an emotional state conveyed by a face of the biological subject;
in response to identifying the emotional state as a emotion indicative of depression in the biological subject, requested electroencephalogram data for the biological subject;
receiving, from the biological subject, the electroencephalogram data;
analyzing the electroencephalogram data, via a second machine learning model, to identify a severity level of depression in the biological subject; and
providing the emotional state identified by the first machine learning model and the severity level identified by the second machine learning model to a graphical user interface.

10. The system of claim 9, wherein the emotional state is identified as one of the group consisting of:
anger;
disgust;
fear;
happiness;
sadness; and
neutral.

11. The system of claim 9, wherein the emotional state identified by the first machine learning model and the severity level identified by the second machine learning model are provided to a graphical user interface in real-time.

12. The system of claim 9, wherein the image data are provided as one of still images or video images.

13. The system of claim 9, the operations further comprising:
generating a treatment plan for the biological subject based on the severity level identified by the second machine learning model.

14. The system of claim 9, wherein the severity level of depression is identified as one of the group consisting of:
not depressed;
mildly depressed;
moderately depressed; and
severely depressed.

15. A non-transitory memory storage device, including instructions that, when executed by a processor perform operations including:
receiving image data of a biological subject under examination for depression;
analyzing the image data, via a first machine learning model, to identify an emotional state conveyed by a face of the biological subject;
in response to identifying the emotional state as a emotion indicative of depression in the biological subject, requested electroencephalogram data for the biological subject;
receiving, from the biological subject, the electroencephalogram data;
analyzing the electroencephalogram data, via a second machine learning model, to identify a severity level of depression in the biological subject; and
providing the emotional state identified by the first machine learning model and the severity level identified by the second machine learning model to a graphical user interface.

16. The device of claim 15, wherein the emotional state is identified as one of the group consisting of:
anger;
disgust;
fear;
happiness;
sadness; and
neutral.

17. The device of claim 15, wherein the emotional state identified by the first machine learning model and the severity level identified by the second machine learning model are provided to a graphical user interface in real-time.

18. The device of claim 15, wherein the image data are provided as one of still images or video images.

19. The device of claim 15, the operations further comprising:
generating a treatment plan for the biological subject based on the severity level identified by the second machine learning model.

20. The device of claim 15, wherein the severity level of depression is identified as one of the group consisting of:
not depressed;
mildly depressed;
moderately depressed; and
severely depressed.
